(19) United States
(12) Patent Application Publication
(10) Pub. No.: US 2026/0258096 A1
(43) Pub. Date: Sep. 3, 2026

(54) FUNCTIONALIZED RECOMBINANT BACTERIAL COLLAGEN-LIKE PROTEINS

(71) Applicant: Evonik Operations GmbH, Essen (DE)

(72) Inventors: Sven WEBER, Dossenheim (DE); Maria MONTERO MIRABET, Mühltal (DE); Ute SCHEPERS, Troisdorf (DE)

(73) Assignee: Evonik Operations GmbH, Essen (DE)

(21) Appl. No.: 19/163,229

(22) PCT Filed: Mar. 8, 2024
(86) PCT No.: PCT/EP2024/056139

§ 371 Date: Sep. 8, 2025

(30) Foreign Application Priority Data
Mar. 16, 2023 (EP) 23162211

(51) Int. Cl. C07K 14/315; A61L 27/24; A61L 27/52; B33Y 70/00; B33Y 80/00; C08J 3/075; C08J 3/24

(52) U.S. Cl. CPC C07K 14/315; A61L 27/24; A61L 27/52; B33Y 70/00; B33Y 80/00; C08J 3/075; C08J 3/24; C08J 2389/00; C08J 2401/02

(57) ABSTRACT

Recombinant bacterial collagen-like protein, preferably with an amino acid sequence that is at least ≥60% identical to the amino acid sequence of SEQ ID NO:1 wherein the amino acid sequence comprises a deletion of at least 38 amino acids at the N-terminus of the amino acid sequence of SEQ ID NO:1, wherein the recombinant collagen-like protein is functionalized with at least one non-terminal alkene group. A bioink comprising the functionalized recombinant bacterial collagen-like protein, at least one solvent and at least one photoinitiator, a process for producing a hydrogel by crosslinking the functionalized recombinant bacterial protein or the bioink as well as the hydrogel obtained therefrom. Furthermore, a scaffold for tissue engineering comprising the hydrogel.

### FIELD OF THE INVENTION

The invention relates to functionalized recombinant collagen-like proteins (CLPs) of bacterial origin, their application as a bioink for cell encapsulation, as well as preparation of 3D scaffolds, in vitro models and tissue grafts.

### BACKGROUND OF THE INVENTION

Bioinks which are commonly used for bioprinting, are largely synthetic thermoplastic polymers or animal-derived proteins e.g., collagen, gelatin, (meth)acryloyl functionalized animal derived collagen- or gelatin methacrylate.

Collagen-based hydrogels are optimal scaffold materials for cell encapsulation. Their biodegradability and biocompatibility allow optimal material conditions for bioprinting while a multiplicity of cell interaction domains (naturally present in collagen) allow cell spreading and scaffold colonization.

US2016/0051727 discloses a collagen based polymeric material comprising collagen molecules and/or collagen derived molecules which are functionalized by the addition of one or more ethylenically unsaturated groups and which are cross-linked via said groups.

US 2020/0179562 discloses curable recombinant human collagen functionalized with methacryloyl, which can be formulated into inks for additive manufacturing and printed into 3 dimensional objects.

US 2016/0193384 discloses the production of a hydrogel by photocrosslinking a methacryloyl or acryloyl collagen and a synthetic polymer. Such hydrogels can be used as 3D scaffolds and implants and they are suitable as inks for 3D printing in order to prepare complex 3D structures, or for incorporating cells into the structure.

Photocurable gelatin-based hydrogels have established themselves as powerful bioinks in tissue engineering due to their excellent biocompatibility, biodegradability, light responsiveness, thermosensitivity and bioprinting properties. While gelatin methacryloyl (GelMA) has been the gold standard for many years, thiol-ene hydrogel systems based on norbornene-functionalized gelatin (GelNB) and a thiolated crosslinker have recently gained increasing importance (Göckler et al. 2021). Göckler et al. disclose a highly reproducible water-based synthesis of GelNB covering a broad range of degrees of functionalization (DoF: 20% to 97%) as well as its mixing with thiolated gelatin (GelS) resulting in the superfast curing photoclick hydrogel GelNB/GelS.

However, the collagens used in the above referenced prior art are obtained from human or animal sources. Such human or animal-derived collagens are not well defined due to their natural origin, are viscous and have poor aqueous solubility under physiological conditions. Due to their viscosity, significant force is needed to extrude or jet these collagen mixtures during the printing process. This results in greater shear stress and thus lower cell viability if cells are co-printed in the same mixture. The low pH-value needed to solubilize animal-derived collagen also makes it challenging to increase the concentration. A further disadvantage due to the origin is the varying quality as well as the risk of disease transmission.

Collagen-like proteins (CLPs) of bacterial origin have interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain (CL). After the collagen domain, typically a membrane anchor (GPI-like) is present at the C-terminal end of the protein.

The most industrially relevant CLP is Scl2 of Streptococcus pyogenes. As described in various publications (Lukomski et al. 2002, Brodsky et al. 2009) the current understanding is that the V-domain is required for folding three Scl2 protein monomers into one triple helical structure in vitro.

Whilst bioinks based on human or animal-derived collagens are widely used, there is still an unmet need for GMP compliant bioinks to facilitate the translation of bioprinted clinical products from bench-to-bedside, particularly water-soluble biomaterials which can form hydrogels and thus support cell encapsulation and bioprinting. Required are properties such as a constant quality of produced batches, high purity as well as good solubility in conditions that provide for a suitable environment for the encapsulated cells.

It is therefore an object of the present invention to provide a protein that can be formulated into a bioink and crosslinked into a hydrogel and which can at least reduce the disadvantages of prior art materials in particular animal derived collagens.

### SUMMARY OF THE INVENTION

The present invention therefore relates in one aspect to a recombinant bacterial collagen-like protein, preferably comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1, wherein the recombinant collagen-like protein is functionalized with at least one non-terminal alkene group.

In one embodiment of the present invention the amino acid sequence is at least 60%, preferably at least 70%, more preferably at least 80%, most preferred at least 90% identical to the amino acid sequence of SEQ ID NO:2, SEQ ID NO:3 or SEQ ID NO:4.

In a further embodiment the degree of functionalization with said at least one non-terminal alkene group ranges from 5% to 100% of the sum of primary amines comprising the N-terminal primary amine group and the primary amine groups of the lysine residues of the whole recombinant collagen-like protein.

In one embodiment of present invention the at least one non-terminal alkene is selected from linear, branched or cyclic non-terminal alkenes, preferably from norbornene or its derivatives, or combinations thereof.

In a further aspect the present invention relates to a bioink composition comprising
a) from about 0.25 to about 20 wt.-% of the at least one functionalized recombinant bacterial collagen-like protein according to the present invention;
b) from about 75 to about 99 wt.-% of aqueous solvent;
c) from about 0.01 to about 2 wt.-% of at least one photoinitiator;
d) from about 0 to about 5 wt.-% of at least one photocrosslinkable polymer or other photocrosslinkable peptides;
e) from about 0 to about 15 wt.-% of at least one compound selected from additives, rheology modifiers, biopolymers, gelation enhancers, bioactive moieties, peptides, nanocellulose and/or cells;
provided that the sum of all components of the bioink amounts to 100 wt.-%.

In one embodiment of present invention the bioink composition further comprises from about 0.125 to about 10 wt.-% of at least one further recombinant bacterial collagen-like protein, wherein said further recombinant bacterial collagen-like protein comprises a functionalization that is different from the functionalization of the functionalized recombinant bacterial collagen-like protein of a).

In a further embodiment of present invention the said at least one further functionalized recombinant bacterial collagen-like protein is a recombinant bacterial collagen-like protein comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO: 1 and wherein the recombinant collagen-like protein is functionalized with at least one thiol group.

In one embodiment of present invention the said at least one further functionalized recombinant bacterial collagen-like protein is a recombinant bacterial collagen-like protein comprising an amino acid sequence that is at least 60%, preferably at least 70%, more preferably at least 80%, most preferred at least 90% identical to the amino acid sequence of SEQ ID NO:2, SEQ ID NO:3 or SEQ ID NO:4.

In one embodiment of the bioink composition of present invention the degree of functionalization of the said further functionalized recombinant bacterial collagen-like protein ranges from 5% to 100% of the sum of primary amines comprising the N-terminal primary amine and the primary amine groups of the lysine residues of the recombinant collagen-like protein.

In one embodiment the bioink composition further comprises a thiol containing molecule, a thiol dimer or a thiol-multimer, preferably a multiarm PEG such as 4-ArmPEG-SH or Dithiothreitol (DTT).

In a further aspect the present invention relates to a process for producing a hydrogel by crosslinking, preferably photocrosslinking, the functionalized recombinant bacterial collagen-like protein or the bioink composition according to the present invention.

In one embodiment of the process of present invention the bioink composition is photocrosslinked using UV light, or visible light, preferably blue light.

In yet a further aspect the present invention relates to a hydrogel obtained by the process according to the present invention, preferably an injectable hydrogel.

In one embodiment of the hydrogel of present invention the hydrogel further comprises nanocellulose, peptides or mixtures thereof.

In yet a further aspect the present invention relates to a scaffold for tissue engineering comprising the hydrogel according to the present invention.

### FIGURES

FIG. 1: Reaction scheme of the modification of the recombinant bacterial collagen-like protein of present invention with a norbornene group resulting in a recombinant bacterial collagen-like protein comprising a norbornene group (rColN) of present invention.

FIG. 2: ¹H-NMR example of the functionalized recombinant bacterial collagen-like protein comprising a norbornene group (rColN) of present invention in comparison to unmodified recombinant bacterial collagen-like protein (CLP).

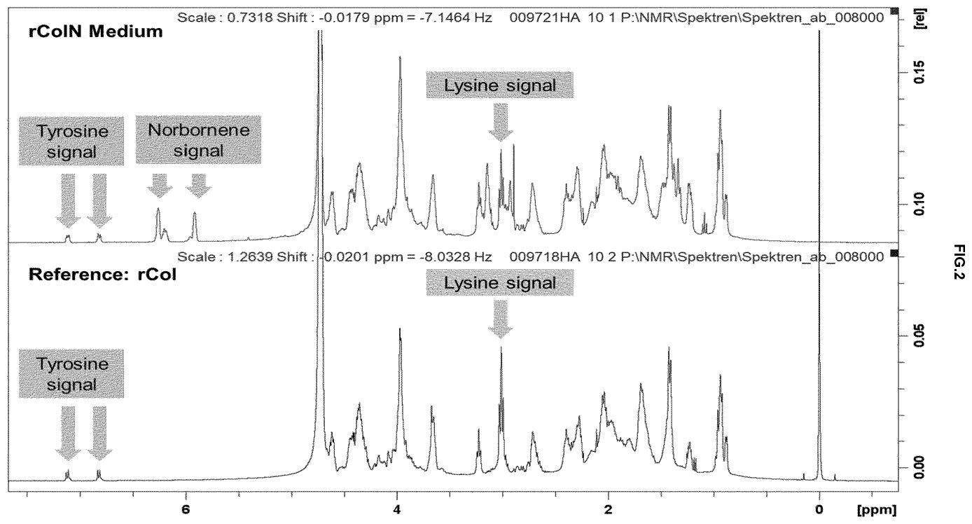

FIG. 3: 3T3 cell viability after exposure to different concentrations of unmodified CLP and rColN after 24 h exposure time. The applied molar ratios equal the following DoFs in %: 1:0.1=15%; 1:0.25=31%; 1:0.5=52%; 1:0.75=76% and 1:1=91%.

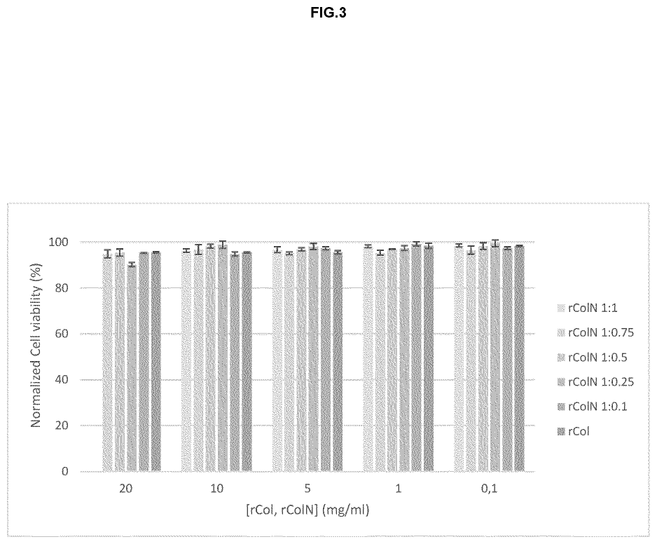

FIG. 4: Reaction scheme of the light-induced crosslinking reaction of the functionalized recombinant bacterial collagen-like protein comprising a thiol group (rColS) and the further recombinant bacterial collagen-like protein comprising a norbornene group (rColN) of present invention with the photoinitiator LAP (lithium phenyl-2,4,6-trimethylbenzoylphosphinate).

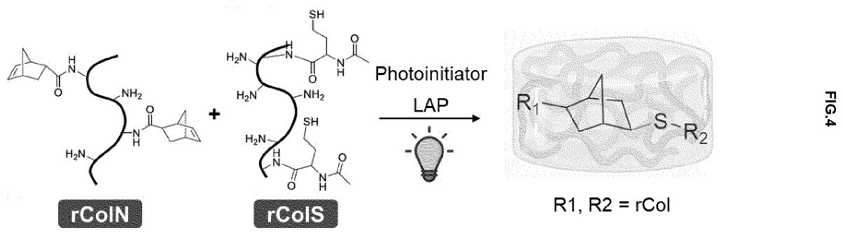

FIG. 5: Stained human dermal fibroblasts (HFF cells) after cultivation for 7 and 14 days inside of rColN hydrogels. A cell network has formed. Nearly 100% of all cells were determined as viable.

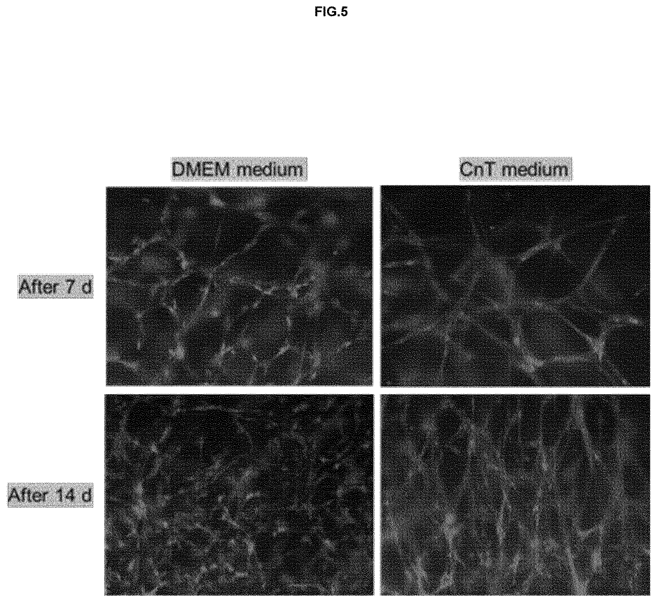

FIG. 6: (A) Printing of rColN mixture described in the text using different technologies. (B) Printing of rColN mixture (1% (w/v) rColN (48%), 1% (w/v) rColS (45%) and 0.03% LAP in 1×PBS printed via Drop-on-Demand.

FIG. 7: Reaction scheme for hydrogel synthesis using rColN and 4-Arm-PEG-SH, 10 kDa (JenKem Technology USA).

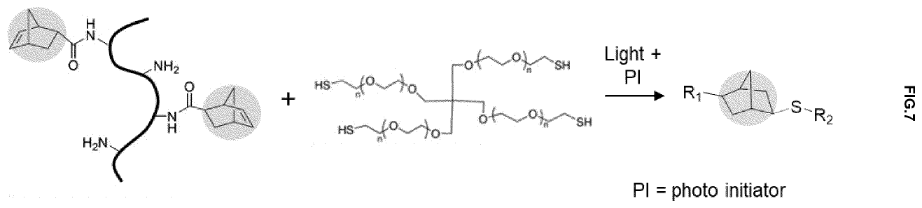

FIG. 8: Correlation between different CLP and rColN concentrations dissolved in 1×PBS and the measured viscosity using an Anton Paar MCR 502 WESP system with a 50 mm plate-cone system (1° angle; N=3). rColN_high equals a DoF of 77% while rColN_low equals a DoF of 21%.

FIG. 9: Example measurement of the shear storage modulus for the online measurement using ElastoSens Bio.

### SEQUENCES

| | |
|---|---|
| SEQ ID NO: 1 | Streptococcus pyogenes Collagen-like protein (CLP), full |
| | length protein |
| SEQ ID NO: 2 | Streptococcus pyogenes CLP, truncation 3 |
| SEQ ID NO: 3 | Streptococcus pyogenes CLP, truncation 5 |
| SEQ ID NO: 4 | Streptococcus pyogenes CLP, no V-domain |

### DETAILED DESCRIPTION OF THE INVENTION

Collagen-based hydrogels are optimal scaffold materials for cell encapsulation. Their biodegradability and biocompatibility allow optimal material conditions for bioprinting while a multiplicity of cell interaction domains (naturally present in collagen) allow cell spreading and scaffold colonization. The currently available and described collagen-based hydrogels are based on human or animal sources. Such human or animal-derived collagens have various disadvantages such as varying quality, high viscosity, poor aqueous solubility under physiological conditions as well as the 30 potential for carrying diseases. Due to their viscosity, significant force is needed to extrude or jet the viscous collagen mixture during the printing process. This results in greater shear stress (and thus lower cell viability) if cells are co-printed in the same mixture. Furthermore, the low pH-value needed to solubilize animal-derived collagen makes it challenging to increase the concentration.

As there is a high need for optimized bioinks that overcome the above disadvantages, the inventors have set out to provide a recombinant collagen-like protein that is of bacterial origin and thus a vegan alternative to the animal-derived collagen materials of the prior art and that can be functionalized and used to prepare optimized bioinks for cell-encapsulation and hydrogel production.

### Recombinant Collagen-Like Protein (CLP)

The most industrially relevant CLP of Streptococcus pyogenes Scl2 contains a large V-domain which makes up for approximately one third of the whole sequence of Scl2 and hinders the protein to be transported out of the Pichia pastoris host. This requires a complex downstream process containing cell lysis to remove the target protein from the cell. Furthermore, the V-domain itself has pathogenic properties and thus needs to be removed during the purification process by protease digestion. Usage of a protease is quite costly, and it needs to be removed during downstream processing. The production of a purified Scl2 starting from a full-length protein including the V-domain thus requires additional costly downstream process steps.

The inventors have now surprisingly found that truncated variants of the collagen-like protein, including variants with a truncated V-domain or without any V-domain lead to increased production of collagen-like protein and secretion into the fermentation medium. It was further surprising that the truncated variants were correctly folded even in absence of the V-domain.

Other than previously described in the prior art the V-domain thus does not seem to be required for the correct folding of the three Scl2 protein monomers into one triple helical structure in vitro, which opened up the possibility to overcome the various challenges caused by expressing the full lengths Scl2 protein first and removing the V-domain later.

The present invention therefore relates in one aspect to a recombinant bacterial collagen-like protein, preferably comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1, wherein the recombinant collagen-like protein is functionalized with at least one non-terminal alkene group.

In one aspect it is preferred that the recombinant bacterial collagen-like protein comprises a deletion of between 38 to 90 amino acids at the N-terminus of the amino acid sequence as shown in SEQ ID NO: 1, where SEQ ID NO: 1 depicts the amino acid sequence of the full-length CLP. Preferred is the deletion of between 38 to 74 amino acids. This includes a complete deletion of the N-terminal V-domain (comprising 74 amino acids) and different truncations of the V-domain of at least 38 amino acids.

The amino acid sequence of the recombinant bacterial collagen-like protein of present invention is preferably at least 60%, identical to the amino acid sequence of SEQ ID NO:2, SEQ ID NO:3 or SEQ ID NO: 4. SEQ ID NO:2 and SEQ ID NO:3 are truncated versions of Scl2 in which amino acids have been deleted as follows:
SEQ ID NO:2 is based on SEQ ID NO. 1 with a deletion of amino acids at position 13-50 (38 aa)
SEQ ID NO:3 is based on SEQ ID NO. 1 with deletion of amino acids at position 1-74 (75 aa)
SEQ ID NO:4 is based on SEQ ID NO. 1 with deletion of aa 1-90 (90 aa)

It is to be understood that the SEQ ID NOs indicated herein describe the amino acid sequence as such before functionalization with the at least one non-terminal alkene group and not the functionalized recombinant bacterial collagen-like protein.

In a preferred embodiment the recombinant bacterial collagen-like protein comprises an amino acid sequence that is at least 90%, 91%, 92%, 93%, 94%, 95%, 96% identical to the amino acid sequences of SEQ ID NO:2, SEQ ID NO:3 or SEQ ID NO:4. Preferably the recombinant bacterial collagen-like protein comprises an amino acid sequence that is at least 97%, more preferably at least 98%, most preferred at least 99% identical to the amino acid sequences of SEQ ID NO:2, SEQ ID NO: 3 or SEQ ID NO:4.

In the most preferred embodiment, the recombinant bacterial collagen-like protein consists of amino acid sequences according to SEQ ID NO:2, SEQ ID NO:3 or SEQ ID NO:4.

In a further preferred embodiment other truncated variants of the collagen-like protein from Streptococcus pyogenes are used as a basis for preparing a recombinant bacterial collagen-like protein functionalized with at least one non-terminal alkene group as further described herein above.

The present invention therefore also encompasses variants of the recombinant bacterial collagen-like proteins according to SEQ ID NO:1 to 4, wherein said variants preferably comprise one or more amino acid exchange(s), insertion(s) and/or deletion(s). In a preferred embodiment such variant contains up to 5, up to 4, up to 3, or up to 2, amino acid exchanges, insertions and/or deletions.

In another preferred embodiment of the present invention the variant of the recombinant bacterial collagen-like protein comprises an amino acid sequence that is at least 60%, 61%, 62%, 63%, 64%, 65%, 66%, 67%, 68%, 69%, preferably at least 70%, 71%, 72%, 73%, 74%, 75%, 76%, 77%, 78%, 79%, more preferably at least 80%, 81%, 82%, 83%, 84%, 85%, 86%, 87%, 88%, 89%, most preferred at least 90%, 91%, 92%, 93%, 94%, 95%, 96%, 97%, 98%, 99% identical to the amino acid sequence of SEQ ID NO:2, SEQ ID NO:3 or SEQ ID NO:4.

The recombinant bacterial collagen-like proteins (CLP) of present invention can be produced in a process comprising the following steps:
a) fermentation of a bacterial, yeast or plant host cell, expressing a CLP with an amino acid sequence that is at least ≥60% identical to the amino acid sequence of SEQ ID NO: 1, in a medium, wherein the amino acid sequence comprises a deletion of at least 38 amino acids at the N-terminus of the amino acid sequence of SEQ ID NO:1,
b) accumulation of the bacterial CLP in the medium, wherein a fermentation broth is obtained,
c) separating the bacterial, yeast or plant host cells from the fermentation broth,
d) incubating the fermentation broth for at least 1 h at not more than 25° C. for folding of the CLP,
e) optionally purification of the bacterial CLP by at least one of the following: solvent precipitation, tangential flow filtration (TFF), ion exchange chromatography, reversed-phase chromatography.

In a preferred embodiment the folding of CLP in step d) is performed at a temperature between −80° C. and 25° C., preferably between 0° C. and 20° C. In a preferred configuration folding is performed in presence of glycerin or salts.

In another preferred embodiment, folding of CLP in step d) is performed for a time between 1 h and 48 h, preferably between 1 h and 24 h.

In another preferred embodiment, folding of CLP in step d) is performed with a concentration of CLP of at least 1 mg/ml, preferably at least 4 mg/ml.

In a preferred embodiment, the host cell is a microorganism of the species P. pastoris, E. coli, P. putida or C. glutamicum comprising any of the polypeptides according to the present invention.

In a preferred embodiment, the microorganism is a yeast of the genus P. pastoris or a bacterial cell, preferably E. coli, Corynebacterium or Brevibactetium.

The microorganism may be a microorganism in which the nucleotide sequence encoding the CLP is present in overexpressed form.

### Functionalization

The recombinant bacterial collagen-like protein of present invention and as described herein above is functionalized with at least one non-terminal alkene group. The non-terminal alkene can be selected from linear, branched or cyclic non-terminal alkenes, preferably from cyclic alkenes having 4 to 16 or 5 to 12 carbon atoms, more preferably from norbornene or its derivatives, or combinations thereof.

In a preferred embodiment of the recombinant bacterial collagen-like protein of present invention the non-terminal alkene group is a norbornene group. The term “norbornene”, also referred to as norbornylene or norcamphene, is a cyclohexene ring with a bridging methylene in para-position.

Compared with traditional radical chain polymerizations known from alkene containing biomaterials like animal derived GelMA or ColMA (methacrylated gelatin and collagen), the non-terminal alkene, specifically the norbornene conjugation leads to a bacterial collagen like protein that keeps its great solubility even at higher concentrations. This results in an excellent material for 3D-printing technologies as described further herein below. In summary, due to its low viscosity, the functionalized recombinant bacterial collagen-like protein of present invention offers a unique collagen-like based material for those 3D-Printing technologies that are designed to process low viscous solutions, such as stereolithography (SLA), digital light processing (DLP) and droplet-based bioprinting (Jetting or Drop on Demand). These technologies are highly compatible with the novel synthetic rColN material.

The functionalized recombinant bacterial collagen-like protein according to the invention can be prepared by reacting a recombinant bacterial collagen-like protein as described above with at least one non-terminal alkene, preferably a norbornene. Especially suitable molecules for modification with norbornene are 5-Norbornene-2-NHS Ester, 5-Norbornene-2,3-dicarboxylic anhydride and 5-Norbornene-2-carboxylic acid.

In a preferred embodiment, the functionalized recombinant bacterial collagen-like protein according to the invention can be prepared by reacting a recombinant bacterial collagen-like protein as described above with 5-Norbornene-2-NHS ester (Nor-NHS). EDC-HCl (N-(3-Dimethylaminopropyl)-N′-ethylcarbodiimide hydrochloride) and NHS can be used to activate norbornene acid to gain Nor-NHS. In such a preferred embodiment only the norbornene unit without additional carboxylic acids is introduced into the recombinant bacterial collagen-like protein. This leads to less change in the isoelectric point compared to the reaction product synthesized with 5-Norbornene-2,3-dicarboxylic anhydride which would introduce additional acidic side chains. The pH-value is maintained at 7 to 8, which is less harmful for the protein. With higher pH unwanted side reactions could occur. The reaction maintains a constant RT (25° C.) and no additional cooling to 4° C. is needed. In addition, the starting material does not require to be dissolved in acetic acid including high viscosities but allows dissolution at neutral pH-value within water. The same applies for the reaction product. This is specifically advantageous when cells are to be encapsulated.

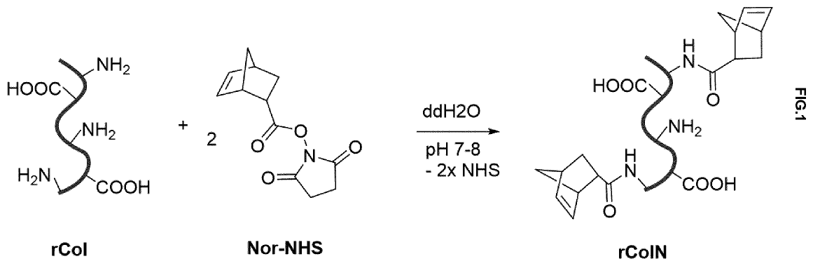

As depicted schematically in FIG. 1, the non-terminal alkene group, specifically the norbornene functional group, is conjugated to primary amine groups present in lysine residues and the N-terminus of the protein.

The degree of functionalization of the recombinant bacterial collagen-like protein can be controlled via adjustment of the molar ratio of primary amines present in the recombinant bacterial collagen-like protein and the norbornene units added, like for example Nor-NHS. Following the functionalization step, the mixture preferably undergoes diafiltration or dialysis to remove side products. The degree of functionalization can be determined by the known methods such as for example a trinitrobenzene sulfonic acid assay, NMR, or HPLC-MS.

Due to the high solubility of the recombinant bacterial collagen-like protein in neutral and alkaline buffers, an unexpectedly high degree of functionalization of up to 100% can be achieved with minimal compromise to the triple helical protein structure.

In a further embodiment the degree of functionalization with said at least one non-terminal alkene group ranges from 5% to 100% of the sum of primary amines comprising the N-terminal primary amine group and the primary amine groups of the lysine residues of the whole recombinant collagen-like protein.

In a preferred embodiment of the invention the degree of functionalization is at least 5%, at least 10%, 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the primary amine group on the N-terminus and the primary amine groups of the lysine residues of the recombinant collagen-like protein.

In another preferred embodiment the degree of functionalization is from 10% to 90%, from 20% to 80%, from 25% to 75%, from 30% to 70%, from 40% to 60%.

As described in further detail in Example 1, the degree of functionalization can be modified by changing the reaction conditions. By modifying the molar ratio of Nor-NHS to —NH₂ groups in recombinant collagen, recombinant collagen with different degree of functionalization (DoF) is obtained. For example, when the ratio of amine groups of recombinant collagen to Nor-NHS was kept at 1:0.25, recombinant collagen with a degree of functionalization of around 31% was synthesized. Increasing the ratio to about 1:0.75 lead to a DoF of 76%.

In comparison, animal-derived collagen typically comprises a degree of functionalization of only about 10 to 20% as extreme reaction conditions denature the triple-helical protein structure, producing gelatin which is less mechanically rigid. The high degree of modification that is obtainable with the functionalized bacterial collagen like protein of present invention is therefore a further advantage over animal-derived collagen.

The different Degrees of functionalization (DoF) allow to adjust the stiffness of the resulting hydrogel produced using the functionalized bacterial collagen like protein of present invention. The material of present invention therefore provides for a much higher variability for multiple applications. A further advantage of the recombinant bacterial collagen-like protein functionalized with a non-terminal alkene group, preferably a norbornene group according to the invention is that it is soluble in water, aqueous buffers at neutral or alkaline pH-value and in particular at physiologically relevant pH-values of 7 to 7.4.

### Bioinks and Hydrogels

To synthesize collagen hydrogels, different crosslinking methods are known from literature for proteins but only a few are suitable for 3D bioprinting.

In a further aspect the present invention relates to a bioink composition comprising
a) from about 0.25 to about 20 wt.-% of the at least one functionalized recombinant bacterial collagen-like protein according to the present invention;
b) from about 75 to about 99 wt.-% of aqueous solvent;
c) from about 0.01 to about 2 wt.-% of at least one photoinitiator;
d) from about 0 to about 5 wt.-% of at least one photocrosslinkable polymer or other photocrosslinkable peptides;
e) from about 0 to about 15 wt.-% of at least one compound selected from additives, rheology modifiers, biopolymers, gelation enhancers, bioactive moieties, peptides, nanocellulose and/or cells;
provided that the sum of all components of the bioink amounts to 100 wt.-%.

The aqueous solvent can be water, any aqueous buffer system, such as for example 1×PBS buffer or HEPES buffer, or also a culture medium. Buffer systems and culture mediums are well known in the prior art and can be chosen according to the required application by the skilled person.

In a preferred embodiment the aqueous solvent is water.

In one embodiment of present invention the bioink composition further comprises from about 0.125 to about 10% weight of a further recombinant bacterial collagen-like protein, wherein said further recombinant bacterial collagen-like protein comprises a functionalization that is different from the functionalization of the functionalized recombinant bacterial collagen-like protein of a).

In a further embodiment of present invention the said further functionalized recombinant bacterial collagen-like protein is a recombinant bacterial collagen-like protein comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO: 1 and wherein the recombinant collagen-like protein is functionalized with at least one thiol group.

The term “thiol group” refers to any organosulfur compound of the form R—SH, where R represents an alkyl or other organic substituent which was designed to react with primary amines. More preferably the recombinant bacterial collagen-like protein is functionalized with a thiolactone group. The term “thiolactone group” refers to analogs of lactones in which an oxygen atom is replaced with a sulfur atom and the sulfur atom is within the ring system adjacent to a carbonyl group. A preferred thiolactone is homocysteine thiolactone and its derivatives, most preferred is N-acetylhomocysteine thiolactone.

In one embodiment of the bioink composition of present invention the degree of functionalization of the said further functionalized recombinant bacterial collagen-like protein ranges from 5% to 100% of the sum of primary amines comprising the N-terminal primary amine and the primary amine groups of the lysine residues of the recombinant collagen-like protein.

In a preferred embodiment of the invention the degree of functionalization ranges from 5% to 90% of the sum of primary amines comprising the N-terminal primary amine group and the primary amine groups of the lysine residues of the whole recombinant collagen-like protein.

In a preferred embodiment of the invention the degree of functionalization is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the sum of primary amine groups of the recombinant collagen-like protein.

In a preferred embodiment the degree of functionalization is from 10% to 90%, from 20% to 80%, from 25% to 75%, from 30% to 70%, from 40% to 60%.

In one embodiment the bioink composition further comprises a thiol containing molecule, a thiol dimer or a thiol-multimer, preferably a multiarm PEG such as 4-ArmPEG-SH or Dithiothreitol (DTT).

In one embodiment of present invention the said at least one further functionalized recombinant bacterial collagen-like protein is a recombinant bacterial collagen-like protein comprising an amino acid sequence that is at least 60%, preferably at least 70%, more preferably at least 80%, most preferred at least 90% identical to the amino acid sequence of SEQ ID NO:2, SEQ ID NO:3 or SEQ ID NO:4.

In a bioink composition the non-terminal alkene, preferably norbornene, groups can form polymeric crosslink networks with functionalized recombinant bacterial collagen-like protein molecules or with other components in the bioink composition in the presence of radicals, anions, nucleophiles or combinations thereof.

The bioink composition of present invention contains a photoinitiator, preferably a free radical photoinitiator. The amount of photoinitiator added to the bioink composition ranges from 0.01 to 2 wt.-%, based on the total weight of the composition. The photoinitiator(s) are capable of producing radicals when irradiated with actinic radiation.

It is to be understood that the addition of at least one photocrosslinkable polymer or other photocrosslinkable peptides (component d)) into the bioink composition of present invention is optional as the functionalized recombinant bacterial collagen-like protein according to the invention can crosslink with itself.

Preferably the photocrosslinkable polymers of component d) are selected from natural (such as hyaluronic acid methacrylate) and synthetic (such as acrylate and methacrylate derivatives of poly(ethylene glycol)) polymers and mixed into the composition to confer additional biological properties (such as anti-fouling) and modulate the physical properties (such as degradability, and swelling behavior in water).

Synthetic peptides with photocrosslinkable groups can be incorporated to confer additional bioactivity such as cell adhesion, stem cell differentiation and enzymatic degradability. Bioactivity broadly describes cell attachment and enzymatic degradation. The bioactivity can be customized via the addition of cell-interactive ligands during the photocrosslinking process.

Photopolymerization or photocrosslinking is preferably triggered by exposure to UV or visible light depending on the photoinitiator chosen. A process for producing a hydrogel by photocrosslinking the functionalized recombinant bacterial collagen-like protein according to the invention also forms part of the invention. Similarly, a process for producing a hydrogel by crosslinking, preferably photocrosslinking, the bioink composition according to the invention also forms part of the invention.

In one embodiment of the process of present invention the bioink composition is photocrosslinked using UV light.

In one embodiment of the process of present invention the bioink composition is photocrosslinked using visible light, preferably blue light (380-500 nm). Preferred is light with a wavelength of 390-410 nm, most preferred 405 nm.

The bioink composition can be formulated by dissolving the functionalized recombinant bacterial collagen-like protein according to the invention in an aqueous solution, adding a water-soluble photoinitiator, such as for example lithium phenyl-2,4,6-trimethylbenzoylphosphinate (LAP) or 1-[4-(2-hydroxyethoxy)-phenyl]-2-hydroxy-2-methyl-1-propane-1-one (Irgacure 2959). Other suitable photoinitiators according to present invention are, 2,2′-Azobis [2-methyl-n-(2-hydroxyethyl)propionamid], tris(2,2bipyridyl)dichlororuthenium (II) hexahydrate, Eosin Y, Ivocerin, ZnTTP and other Irgacure derivates.

Components d) and e) can be optionally included into the composition depending on the end application.

The invention also encompasses a bioink composition comprising 0.25% to 10% (w/w) of the recombinant bacterial collagen-like protein functionalized with non-terminal alkene, preferably norbornene groups and/or the thiolated recombinant collagen-like protein. Within this concentration range, formulations containing 0.5 to 10 wt.-% of total recombinant bacterial collagen-like protein are preferred. Total recombinant bacterial collagen-like protein includes any recombinant bacterial collagen-like protein comprising a functionalization as described herein.

The bioink composition according to the invention has lower viscosity compared with formulations using functionalized animal-derived collagen, and therefore puts less shear stress on the cells, and less chances of the nozzle clogging during bioprinting. In the absence of additives, when dissolved in water or physiological buffers, the viscosity of the bioink composition according to the invention can be between 1 to 200 centipoise. In one embodiment the viscosity of the bioink composition is below 120, below 100, below 80, below 60, below 50, below 40, below 30, below 20, below 10 centipoise.

The bioink composition according to the invention can be applied towards the preparation of photocrosslinked hydrogels and sponges. The invention therefore also pertains to a process for producing a hydrogel by photocrosslinking the functionalized recombinant bacterial collagen-like protein according to the invention. The bioink composition can undergo photocrosslinking to form transparent and colorless hydrogels in the presence of actinic radiation, UV light or visible light (VIS). Here specifically blue light was used.

In a preferred embodiment the light has a wavelength of between 365 nm to 405 nm, due to the applied photo initiator LAP.

The resulting pH-value of the bioink composition and thus the hydrogel is preferably between 6.5 and 8. A hydrogel obtained by the process according to the present invention is also part of the present invention.

The bioink composition of the present invention can be dispensed using a bioprinter or poured into a mold for photocuring to form hydrogel scaffolds.

Another advantage of the bioink composition according to the invention is that the mechanical properties of the resulting hydrogels are significantly more rigid compared to functionalized animal-derived collagen hydrogels and rigidity (as determined by storage moduli measured using rheology) can be adjusted by varying the concentration of the functionalized recombinant bacterial collagen-like protein according to the invention from 0.25 to 10 wt.-% of the bioink composition and/or by choosing alternating DoFs.

In one embodiment of present invention the bioink composition comprises 1.5 to 6 wt.-%, more preferably 3 to 6 wt.-% of functionalized recombinant bacterial collagen-like protein according to the invention. In one embodiment the bioink composition further comprises only at least one thiolated recombinant bacterial collagen-like protein according to the invention. In a further embodiment the bioink composition comprises only a recombinant bacterial collagen-like protein functionalized with norbornene groups according to the invention.

The advantageous mechanical properties of the resulting hydrogels are partly due to the higher solubility of the recombinant collagen itself, enabling the formation of high concentration hydrogels. In comparison, due to solubility considerations, bioinks derived from animal-origin collagen are typically less than 0.6% (rat tail collagen). The rigidity of rColN hydrogels in combination with rColS allow rigidities of 5.7 kPa with only 2% total collagen with ~50% DoF (Degree of functionalization) each. Using 1% total collagen a stiffness of only 1.3 kPa was reached. Using 5% rColN (77% DoF) in combination with 5% rColS (65%) a stiffness of >50 kPa can be reached. It is assumed that higher concentrations lead to an exponential increase in stiffness. Hydrogels comprising a photocrosslinked functionalized recombinant bacterial collagen-like protein according to the invention are therefore also part of the invention.

The additives that can be incorporated into the bioink composition as component e) include rheology modifiers, gelation enhancers and/or bioactive moieties. These additives can enhance the mechanical, viscoelastic and biological properties of the bioink composition and/or the resulting photocrosslinked hydrogel.

Self-assembling peptides and biopolymers such as nanocellulose can also be incorporated in the bioink composition as component e). Such molecules enhance the gelation and confer additional mechanical properties. Therefore, the invention also pertains to a hydrogel comprising a photocrosslinked functionalized recombinant bacterial collagen-like protein according to the invention the hydrogel further comprising nanocellulose, peptides or mixtures thereof.

In a further embodiment the bioink composition comprises as component e) additional components with thiol side chains, such as cysteine containing proteins or drugs. Such additives are useful for customizing the scaffold for specific needs.

In a further embodiment the bioink composition comprises as component e) glycosaminoglycan, e.g. chondroitin sulfate, hyaluronic acid, silk, elastin, keratin, resilin, titin, elastin-like polypeptides, fibrin, fibrinogen, fibronectin, thrombin, chitosan, carbohydrates like dextran or chitin, growth factors, platelet-rich plasma (PRP), cell binding peptides, oligonucleotides like DNA and RNA.

Cells can be encapsulated in the photocrosslinking process by adding them into the bioink composition. Cells can also be subsequently incorporated into the photocrosslinked hydrogel scaffolds. Whether printed or cast, a scaffold for tissue engineering comprising the hydrogel above is also subject matter of the present invention.

The resulting photocrosslinked hydrogels support the proliferation of different types of cells in vitro. Cells can advantageously also be incorporated into the bioprinting process. The cell viability, proliferation and spreading in bioprinted hydrogels are significantly better than in bulk-casted hydrogels. Thus, the photocrosslinked hydrogels are especially suited as scaffolds for tissue engineering.

The hydrogels formed from the bioink composition according to the invention, either bioprinted or cast, demonstrate good stability in vivo exceeding three months compared to animal-derived collagen which was resorbed by the natural tissues within a month.

The bioink composition according to the invention can be formulated for different 3D printing or bioprinting technologies, particularly Drop-on-Demand/jetting and digital light printing/stereolithography. The bioink is particularly suited for Drop-on-Demand printing due to its low viscosity.

The bioink is also suited towards digital light printing, where bioink compositions containing 1.5 to 6 wt.-% of functionalized recombinant bacterial collagen-like protein according to the invention have been successfully printed into 3D hydrogel constructs.

Preferably bioink compositions containing 1.5 to 6 wt.-% of functionalized recombinant bacterial collagen-like protein according to the invention, more preferably 3 to 6 wt.-% can be printed into 3D hydrogel constructs. In combination with a thiol comprising compound, like the said further functionalized recombinant bacterial collagen-like protein, which is a recombinant bacterial collagen-like protein comprising an amino acid sequence that is at least 60% identical to the amino acid sequence of SEQ ID NO: 1, wherein the amino acid sequence comprises a deletion of at least 38 amino acids at the N-terminus of the amino acid sequence of SEQ ID NO:1 and wherein the recombinant collagen-like protein is functionalized with at least one thiol group, as described in the invention, the needed concentration for 3D printing can be lowered significantly.

In a further aspect the present invention relates to a process for producing a hydrogel by crosslinking, preferably photocrosslinking, the functionalized recombinant bacterial collagen-like protein or the bioink composition as described herein.

In yet a further aspect the present invention relates to a hydrogel comprising a photocrosslinked functionalized recombinant bacterial collagen-like protein and/or a further functionalized recombinant bacterial collagen-like protein as described herein.

In one embodiment of the hydrogel of present invention the hydrogel further comprises nanocellulose, peptides or mixtures thereof.

In yet a further aspect the present invention relates to a scaffold for tissue engineering comprising the hydrogel as described herein.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

“At least one”, as used herein in relation to any component, refers to the number of chemically different molecules or groups, i.e. to the number of different types of the referenced species, but not to the total number of molecules or groups in the composition or compound. For example, “at least one non-terminal alkene group” means that at least one type of non-terminal alkene groups is used but that also two or more different types of non-terminal alkene groups can be present, but does not mean that only one or more groups (number) of non-terminal alkene groups are present.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by +1, 2, 3, 4, 5, 6, 7, 8, 9, or 10%. Accordingly, the term “about” may mean the indicated value±5% of its value, preferably the indicated value±2% of its value, most preferably the term “about” means exactly the indicated value (±0%).

The following examples serve to illustrate the present invention and should not be construed as limiting the scope thereof.

### EXAMPLES

### Example 1: Preparation of the Functionalized Recombinant Bacterial Collagen-Like Protein with Different Degrees of Functionalization (DoF)

The norbornene-functionalized CLP (rColN) was synthesized with Vecollan, a recombinant Scl2 based CLP from Evonik Operations GmbH and 5-Norbornene-2-NHS Ester (short: NCA-NHS; BroadPharm; BP-24407). All subsequent steps were executed as sterile as possible.

A clear 4% (w/v) rCol solution in 0.1 M HEPES buffer pH 8.0 was prepared at RT (20-25° C.) on an orbital shaker (450 rpm) overnight. Different masses of NCA-NHS were filled in clean and sealable glass vials followed by collagen solution. The covered reaction was stirred for 24 h at RT. Occurring flocculation disappeared within the reaction time and represented an indicator for the reaction progress. The solution was diluted 1:2 with reaction buffer followed by dialysis (cellulose tubing; cut-off: 12-14 kDa) for 3 days against alkalic ddH₂O (PH~8.0) at RT (20-25° C.). Water was exchanged regularly (every 1-2 h; at least 3× per day). The purified product was freeze dried to generate a white sponge-like material which was stored at 4° C. for further usage. Optionally, the product can be sterile filtrated using a 0.2 μm pore size filter before lyophilization to ensure material sterility.

By altering the Nor-NHS ratio, the degree of functionalization (DoF) changes. In the attached examples DoM of 15%-91% were synthesized. The degree DoF was calculated from ¹H-NMR spectra.

The used collagen-like protein contains one tyrosine, whose independent signal can be used to adjust the overall signal intensity. The degree of functionalization (DoF) was quantified from the peak area ratio of the norbornene signal (5.83 to 5.98 ppm and 6.10 to 6.28 ppm) and the tyrosine signal (6.77 to 6.85 ppm and 7.06 to 7.15 ppm) by considering the number of hydrogen atoms which belong to C═C double bonds and which are responsible for each signal. Each collagen strand contains one tyrosine side chain and 23 primary amines (22 lysine side chains and one N-terminal primary amine).

$$DoF ( \% ) = \frac{A ( 5.83 to 5.98 ppm and 6.1 to 6.28 ppm ) / 2}{A ( 6.77 to 6.85 ppm and 7.06 to 7.15 ppm ) / 4} * \frac{1}{23} * 100$$

TABLE 1
Example DoF of different rCoIN synthesis trials.

| m (rCol) (mg) | m (Nor-NHS) (mg) | DoF (%) |
|---|---|---|
| 200 | 4.7 | 15 |
| 200 | 11.8 | 31 |
| 200 | 23.7 | 52 |
| 200 | 35.7 | 76 |
| 200 | 47.4 | 91 |

### Example 2: Material Safety-Cell Viability of Dissolved and Modified CLP

3T3 mouse fibroblast cells were pre-seeded with 1×10⁴ cells/well (1×10⁵ cells/ml, 0.1 ml) into a transparent 96 well plate. 20 mg/ml sample stock solutions in culture medium were diluted with culture medium for the following concentrations: 20, 10, 5, 1, 0.1 mg/ml. After 14-24 h incubation under culture conditions, the culture medium was replaced by 100 μl test solution each. Each formulation was tested in triplicate. As background, the formulation was tested without cells with 100 μl each. Fresh medium with cells was used for the negative and positive control. Cells were exposed to the test formulations for 24 h under cell culture conditions. Then, the test solution was replaced by 100 μl fresh medium+20 μl of premixed MTS reagent solution per well (CellTiter 96® AQueous Non-Radioactive Cell Proliferation Assay (Promega)) resulting in a total volume of 120 μl per well. 10 min before adding the MTS solution 5 μl Triton X-100 (20% (v/v)) was added to the positive control. The test plate was incubated for 4 h at 37° C. under culture conditions. Then, the absorbance was recorded at 490 nm. The average signal of the positive control was subtracted from each test sample and the sample signals were normalized on the negative control. The average of the sample values as well as the standard deviation was calculated.

### Example 3: Hydrogel Preparation without Cells with rColN

100 mg/ml rColN was dissolved in 1×PBS until a clear solution formed. 10 mg/ml Lithium phenyl-2,4,6-trimethylbenzoylphosphinate (LAP) was dissolved in 1×PBS for at least 2 h prior the experiment. 1×PBS, rColN_{(aq)}, and LAP_{(aq)} were mixed in a suitable vessel and exposed to light of a wavelength of 365-405 nm. Depending on the DoF and the applied concentrations of rColN and LAP, different irradiation times were needed. To show the cruciality of concentrations samples in table 2 were tested for gelation.

TABLE 2
Gelation trials with rCoIN and LAP.

| 0.3 mg/ml LAP | | | | | | | | 1 mg/ml LAP | | | | | | | |
|---|---|---|---|---|---|---|---|---|---|---|---|---|---|---|---|
| [rCoIN] | DoF (rCoIN) (%) | | | | | | | [rCoIN] | DoF (rCoIN) (%) | | | | | | |
| (mg/ml) | 8 | 12 | 29 | 41 | 48 | 56 | 80 | (mg/ml) | 8 | 12 | 29 | 41 | 48 | 56 | 80 |
| 5.0 | X | X | X | X | X | X | X | 5.0 | X | X | X | X | X | X | X |
| 10 | X | X | X | X | X | X | X | 10 | X | X | X | ✓ | ✓ | ✓ | X |
| 20 | X | X | X | X | X | X | X | 20 | X | X | ✓ | ✓ | ✓ | ✓ | ✓ |
| 40 | X | X | X | X | X | X | X | 40 | ✓ | ✓ | ✓ | ✓ | ✓ | ✓ | ✓ |

The total collagen concentration is indicated in the first row.
(left) A LAP concentration of 0.3 mg/ml was used for all formulations.
(right) A LAP concentration of 0.3 mg/ml was used for all formulations.
1x PBS was used as solvent.
All samples were irradiated with the same intensity.
Successful gelation (✓) and insufficient gelation (X) was documented.

To show the advantage of thiolene chemistry using rColN, the material was combined with rColS (thiolated recombinant collagen-like protein as described herein) in identical masses.

TABLE 3
Gelation trials with rCoIN, CoIS and LAP. rCoIN and
CoIS were always used with identical concentrations.

| | DoF | DoF | [total Collagen] (mg/ml) (1:1 mixture of rCoIN | | | | |
|---|---|---|---|---|---|---|---|
| Bioink | (rCoIN) | (rCoIS) | and rCoIS) | | | | |
| Classification | (%) | (%) | 2.5 | 5.0 | 10 | 20 | 40 |
| Extra low | 8 | 8 | X | X | X | X | ✓ |
| Extra low - low | 12 | 17 | X | X | X | ✓ | ✓ |
| Low | 29 | 32 | X | ✓ | ✓ | ✓ | ✓ |
| Medium | 41 | 45 | ✓ | ✓ | ✓ | ✓ | ✓ |
| Medium | 48 | 45 | ✓ | ✓ | ✓ | ✓ | ✓ |
| high | 80 | 81 | ✓ | ✓ | ✓ | ✓ | ✓ |

The total collagen concentration is indicated in the first row.
A LAP concentration of 0.3 mg/ml was used for all formulations.
1x PBS was used as solvent.
All samples were irradiated with the same intensity.
Successful gelation (✓) and insufficient gelation (X) was documented.

For optimal mixing to achieve a homogenous solution, the acellular mixture was vortexed. Due to the low viscosity and lack of close pH-limitations or temperature sensitivity, it was considerably easier to obtain homogenous solution and subsequent hydrogel compared to animal-derived collagen.

### Example 4: Material Suitability for Great Cell Stretching Inside of Hydrogels Made of rColN

10 mg/ml rColN, 2.5 mg/ml 4-Arm-PEG-SH (10 kDa, JenKem Technology USA) and 0.3 mg/ml LAP (lithium phenyl-2,4,6-trimethylbenzoylphosphinate) were mixed in 1×PBS buffer together with HFF cells (P4). The mixture was homogenized and respectively 200 μl sample/well was filled in a sterile 8 well chamber slide. Each well contained 1×10⁵ HFF cells. The formulation was irradiated with light of a wavelength area of 365 nm-405 nm until the hydrogel solidified. Mount every sample with 500 μl culture medium and incubate for 2 weeks. Use 2 types of medium: DMEM high glucose and low serum medium (CnT Fibroblast Growth Medium). Change medium every 2-3 days. After seven and 14 d, a LIVE/DEAD staining (ThermoFisher Scientific) was used to determine viable (green) and dead (red) cells. The cell nuclei were stained with Hoechst 33342 (blue).

### Example 5: 3D Bioprinting

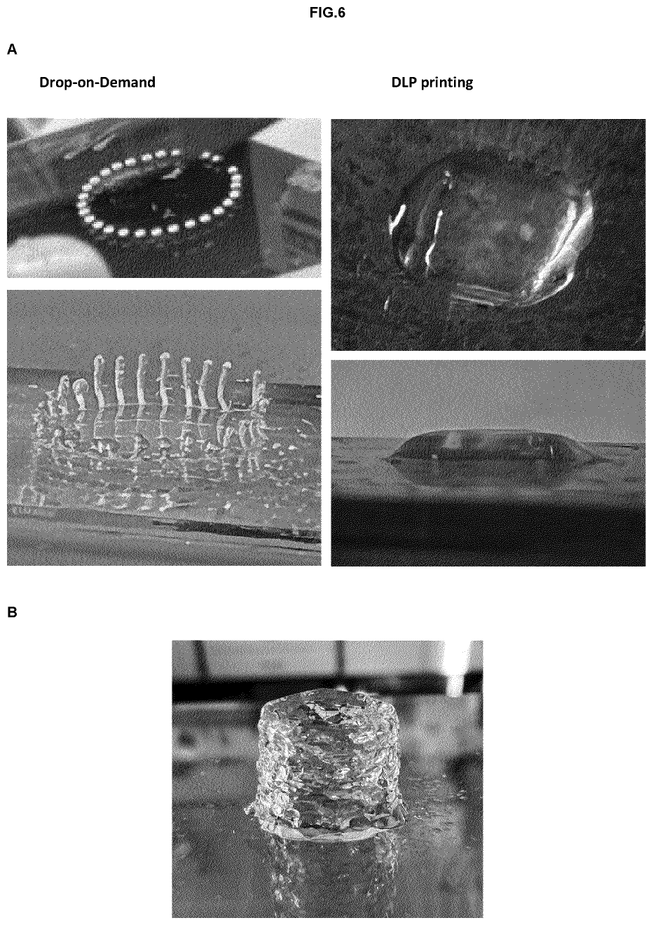

To show the printability of the material with light-processing technologies, 50 mg/ml rColN medium with 50% DoM was dissolved in ddH2O containing 0.2% LAP. With a drop-on demand printer droplets were successfully generated and gelated. Additionally, a flat rectangle was printed using digital light processing (DLP) (FIG. 6).

### Example 6: Viscosity Measurements

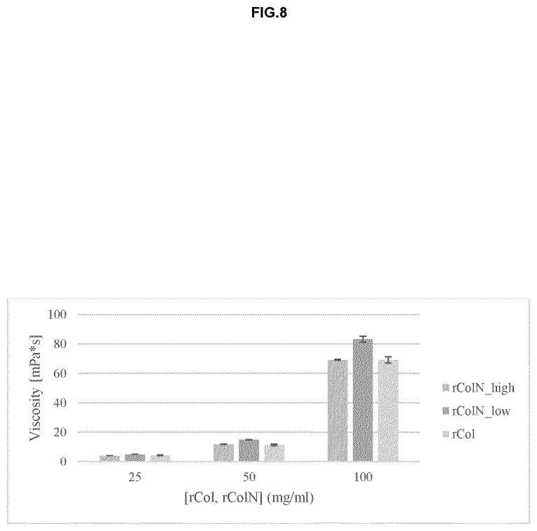

To better understand the viscosity of the material, rheological measurements were performed with the unmodified CLP (rCol). Solutions viscosities were measured using an Anton-Paar MCR 502 WESP system equipped with a plate-cone extension. The lower plate was flat (stainless steel, Ø 50 mm, CP50) while the cupper cone had a 1° angle to the middle (stainless steel, Ø 50 mm, CP50-1) with a truncation of 99 μm. A standard flow curve program was applied. The measurement sequence is described in the following. A sample load of 750 μl was used. The stock solution was made one day in advance and left for complete dissolution on an orbital shaker at 25° C. overnight. Excess sample was removed with a tissue. The measurement was performed at 25° C. with data points and a shear of 0.1-1000. Data extraction: The average of data points in the linear range was calculated for each measurement and the average of three subsequent measurements was calculated along the standard deviation. The results are shown in FIG. 8.

### Example 7: Rheological Measurements

Rheological measurements were performed with the benchtop ElastoSens™ Bio device from Rheolution Live Sciences. The non-destructive non-contact measurement relies on induced vibration of the sample holder silicon bottom whose amplitude is recoded by a laser. The stiffer the formulation, the lesser the response in amplitude.

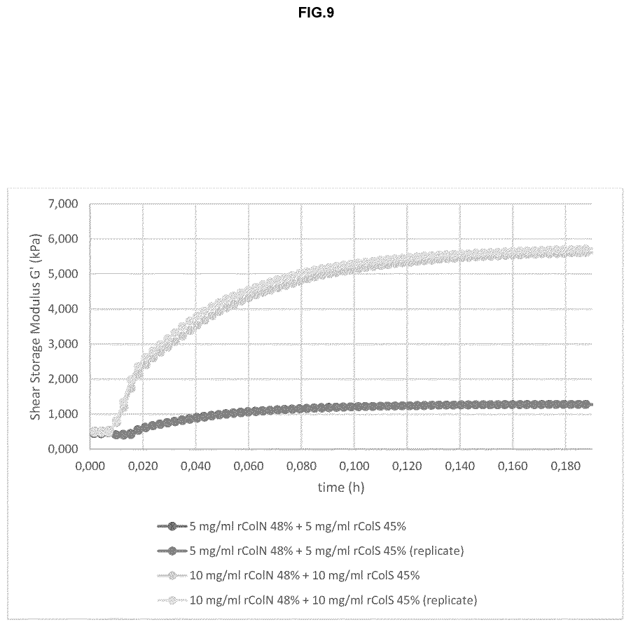

For photopolymerization, hydrogel formulation with 2.2 ml were prepared. 2 ml was transferred into the calibrated sample holder using reverse pipetting. The sample was irradiated with 405 nm (50% lamp power; equals a light power of 11.6 mW/cm² according to the providers light power chart) until a stable Shear Storage Modulus (G′). The stiffness was measured every 10 seconds. The hydrogel height was recorded by the device ensuring no significant decrease. If not stated otherwise, the reaction temperature was kept constant at 25° C. If the recorded Shear Storage modulus (G′) was below 500 Pa using the standard stiff mode, the measurement was repeated with 7 ml of the same formulation according to the user manual in soft mode. The results are shown in FIG. 9.

1. A recombinant bacterial collagen-like protein, comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1, wherein the recombinant collagen-like protein is functionalized with at least one non-terminal alkene group.

2. The recombinant bacterial collagen-like protein according to claim 1, wherein the amino acid sequence is at least 60% identical to the amino acid sequence of SEQ ID NO:2, SEQ ID NO:3 or SEQ ID NO:4.

3. The recombinant bacterial collagen-like protein according to claim 1, wherein a degree of functionalization with the at least one non-terminal alkene group is from 5% to 100% of a sum of primary amines comprising the N-terminal primary amine group and primary amine groups of lysine residues of the recombinant collagen-like protein.

4. The recombinant bacterial collagen-like protein according to claim 1, wherein the at least one non-terminal alkene group is a linear non-terminal alkene group, branched non-terminal alkene group, cyclic non-terminal alkene group, or a combination thereof.

5. A bioink composition, comprising:
a) from 0.25 to 20 wt. % of at least one functionalized recombinant bacterial collagen-like protein of claim 1;
b) from 75 to 99 wt. % of an aqueous solvent;
c) from 0.01 to 2 wt. % of at least one photoinitiator;
d) from 0 to 5 wt. % of at least one photocrosslinkable polymer or a different photocrosslinkable peptide;
e) from 0 to 15 wt. % of at least one compound selected from the group consisting of an additive, rheology modifier, biopolymer, gelation enhancer, bioactive moiety, peptide, nanocellulose and a cell;
provided that the sum of all components of the bioink amounts to 100 wt. %.

6. The bioink composition according to claim 5, where the composition comprises from 0.125 to 10 wt. % of at least one further recombinant bacterial collagen-like protein, wherein the at least one further recombinant bacterial collagen-like protein comprises a functionalization that is different from the functionalization of the functionalized recombinant bacterial collagen-like protein of a).

7. The bioink composition according to claim 6, wherein the the at least one further functionalized recombinant bacterial collagen-like protein is a recombinant bacterial collagen-like protein comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO: 1 and wherein the recombinant collagen-like protein is functionalized with at least one thiol group.

8. The bioink composition according to claim 6, wherein the the at least one further functionalized recombinant bacterial collagen-like protein is a recombinant bacterial collagen-like protein comprising an amino acid sequence that is at least 60% identical to the amino acid sequence of SEQ ID NO:2, SEQ ID NO:3 or SEQ ID NO:4.

9. The bioink composition according to claim 6, wherein a degree of functionalization of the at least one further functionalized recombinant bacterial collagen-like protein is from 5% to 100% of a sum of primary amines comprising the N-terminal primary amine and primary amine groups of lysine residues of the recombinant collagen-like protein.

10. The bioink composition of claim 5, wherein the composition further comprises a thiol containing molecule, a thiol dimer or a thiol-multimer.

11. A process for producing a hydrogel by crosslinking the functionalized recombinant bacterial collagen-like protein of claim 1.

12. A hydrogel obtained by the process of claim 11.

13. The hydrogel according to claim 12, wherein the hydrogel further comprises a nanocellulose, a peptide or a mixture thereof.

14. A scaffold for tissue engineering or tissue regeneration comprising the hydrogel of claim 12.


SEQUENCE LISTING (9 sequences)

SEQ ID NO: 1 - protein, 330 aa, Streptococcus pyogenes
NHKVHMHHHHHHADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 292 aa, Streptococcus pyogenes
NHKVHMHHHHHHDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPAGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 6 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPQGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 7 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPAGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 8 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPSGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 9 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPKGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY